(19) United States
(12) Patent Application Publication
(10) Pub. No.: US 2020/0339918 A1
(43) Pub. Date: Oct. 29, 2020

(54) FRESHENER FOR SPACES AND OBJECTS

(71) Applicant: Oxylus d.o.o., Osijek (HR)

(72) Inventor: Ivo Domazet, osijek (HR)

(21) Appl. No.: 16/764,916

(22) PCT Filed: Nov. 13, 2018
(86) PCT No.: PCT/HR2018/000020

§ 371 Date: May 18, 2020

(30) Foreign Application Priority Data
Nov. 17, 2017 (HR) P20171785

(51) Int. Cl. C11D 3/50; A61L 9/012; A61L 9/14

(52) U.S. Cl. CPC C11D 3/505; B82Y 30/00; A61L 9/14; A61L 9/012

(57) ABSTRACT

The freshener revealed here is used for perfuming and freshening various objects and spaces. It comprises several components, one of which is in the form of a capsule which contains a fragrance. The capsule may be a microcapsule or a nanocapsule. It is applied by spraying directly on the fabric or surface. A pleasant, floral fragrance smells from the first application, however, the specificity of this freshener is noticeable after it is dried on the treated surface. After application by spraying and the time of drying, in physical contact with the treated surface the capsules break and release the fragrance into the space. This freshener is used on all types of textiles, leather, artificial leather, but also on hard and less absorbent surfaces. Mechanic force/friction (sitting, walking and the like) cause the breaking of the fragrant capsules that remain on the fibres, thereby obtaining the additional “boost” of fragrance. By its very nature, the product is completely safe to use, it does not comprise hazardous components. These capsules provide longer lasting freshness because the fragrance is protected as long as the capsules are intact. Only after application of a force, by breaking capsules, pleasant fragrance is released.

### FIELD OF THE INVENTION

The present invention relates to fresheners for spaces and objects and substances which release fragrances, and the application procedure thereof.

### Technical Problem

Usually, the objects that release fragrances are manufactured in such a way that the fragrances are applied onto these objects; afterward, such products are packed in plastic packaging that does not release fragrances, which plastic packaging is removed from these objects during use. Such objects have i.e. release intensive fragrance at the beginning, and, as time goes by, the fragrance intensity is weakened and finally vanishes. The problem lies in the fact that, once the object is taken out of the plastic packaging, it is no longer possible to control neither intensity nor quantity of the releasing fragrance. Hence, the object releases fragrance, whereby the fragrance is spent uncontrollably.

The existing solutions in space freshening are mainly based on devices that occasionally inject a certain fragrant substances into the space, or the fragrant substances are permanently released into the space. However, with these methods there is no control, whether the people for whom the fragrant substance is released into the space, actually are in the space.

With the present invention, the inventor has solved the problem how to save on consumption of fragrant substances in order to be consumed only when persons are present in the space, namely that the object intended to be freshened with fragrance releases fragrance only when in use, i.e. when people use it.

### PRIOR ART

There are products on the market onto which, by special procedures, the fresheners for objects are applied, which fresheners need to be scratched on the surface to release the fragrance; however, since the fragrance carrier or the medium which releases the fragrance is not in the capsule, the scent particles are still slowly leaving in the atmosphere and after a while, such space freshener no longer has its function. The technological procedures used for applying such fresheners for objects are not suitable for personal use.

There are also space fresheners that spray fragrance (perfume) into the space in certain time sequences. However, the problem is that such fragrances are being sprayed irrespectively of the presence of persons. Such fragrances, once they get into the space, quickly lose their scent intensity and need to be added again.

Also, perfumes and cologne waters that are applied to the skin or clothing by spraying or otherwise commonly loose a scent within 24 hours. According to the researches carried out by the inventor, the capsules revealed in the present invention retain the fragrance irrespectively of whether they were exposed to high temperature up to +40° C. or low temperature to −20° C. even several months after being exposed to such weather conditions.

### BRIEF SUMMARY OF THE PRESENT INVENTION

The freshener revealed here is used for perfuming and freshening various objects and spaces. It comprises several components, one of which is in the form of a capsule which contains a fragrance. The capsule may be a microcapsule or a nanocapsule. It is applied by spraying directly on the fabric or surface. A pleasant, floral fragrance smells from the first application, however, the specificity of this freshener is noticeable after it is dried on the treated surface. After application by spraying and the time of drying, in physical contact with the treated surface the capsules break and release the fragrance into the space. This freshener is used on all types of textiles, leather, artificial leather, but also on hard and less absorbent surfaces. Mechanic force/friction (sitting, walking and the like) cause the breaking of the fragrant capsules that remain on the fibres, thereby obtaining the additional “boost” of fragrance. By its very nature, the product is completely safe to use, it does not comprise hazardous components. These capsules provide longer lasting freshness because the fragrance is protected as long as the capsules are intact. Only after application of a force, by breaking capsules, a pleasant fragrance is released.

Freshener for spaces and objects revealed therein comprises capsules which contain a fragrant substance, wherein, when a single capsule breaks, the scent of the fragrant substance is released into the space and wherein the subject fresheners for spaces and objects provide a longer lasting freshness, as the fragrance is protected in each capsule as long as the said capsule remains intact. The fragrant substance in such capsule comprises demineralised water, a thickener or viscosity modifier, sodium chloride and preservative. Usually, the viscosity modifier is a cellulose gum or an acrylic copolymer. Such capsules may either be nano-capsules or micro-capsules. Methods for producing such capsules are either one of the nanoencapsulation methods, or one of the microencapsulation methods.

The essences of this invention in fact are the capsules of fragrance that break by physical contact and release a pleasant fragrance. This freshener is long-lasting and very economical, does not spend over time if it is left untouched, it does not damage any surfaces and does not leave non-washable traces, it is easy to use and apply. It can also be used in washing machines for laundry, carpets and upholstery, it is excellent and adheres well to all types of textiles, leather, artificial leather, as well as on hard and less absorbent surfaces.

Capsules may have, but not necessarily, “release on demand”, “slow release” or “high diffusion” effects, or any of the aforementioned. In the case of “release on demand”, after the application of force, the membrane of the fragrance capsule breaks and the fragrance is released at once. In the case of a “slow release”, after the application of force, the membrane of the fragrance capsule cracks and, after releasing the initial noticeable amount of the fragrance, it continues to release fragrance over a certain period of time (even up to several months). The embodiment marked as the “High Diffusion” means that after the capsule is activated by touch, the capsule has a better/stronger instantaneous release of the fragrance, but also the effect of prolonged action/fragrance release.

The usual manner of use of this freshener for spaces and objects is to apply it by spraying directly onto the surface or the fabric. Such freshener may also be applied to objects by sprinkling onto the surface.

This freshener is especially suitable for freshening of the fabrics and textiles.

### DETAILED DESCRIPTION OF AT LEAST ONE WAY OF CARRYING OUT THE INVENTION

One of the possible methods of preparation of this freshener for spaces and objects is to prepare a fragrant blend comprising 50-100 parts by mass of demineralised water, 0-10 parts by mass of thickener or viscosity modifier, 0-10 parts by mass of sodium chloride, 0-10 parts by mass of nano-encapsulated fragrance and 0-1 parts by mass of preservative.

Afterwards with such a mixture, by nanoencapsulation method, the nano-capsules are made. The resulting mixture i.e. the nanocapsules are mixed with a particular medium and filled into pressurized or non-pressurized vials.

The method of using the subject freshener for spaces and objects is to apply it by spraying directly onto the surface or the fabric. It takes some time for the applied freshener to dry. A fragrance occurs when the nanocapsules break, usually due to pressure or friction which they are exposed to, for example, by walking on the surface comprising nanocapsules, sitting on the chair on the surface of which are the nanocapsules, wiping with the towel with nanocapsules, etc.

This freshener may also be applied by coating, sprinkling or immersion.

After the drying time and water evaporation, a thin layer of nanocapsules with fragrance is formed on the treated surface. Typical applications are in transport and carriage of passengers and goods, tourism, foodservice sector and hospitality industries, public institutions and public facilities, health facilities, sport facilities, wherein the freshener is applied onto the carpets, upholstered furniture and seats, seats and floors in vehicles for passenger transport and transporting goods, on the bed linen, napkins, tablecloths, curtains, towels, upholstery, and clothing.

A commonly used preservative is either 5-chloro-2-methyl-4-isothiazolin-3-one and 2-methyl-4-isothiazolin-3-one and benzyl alcohol or 1,2-benzisothiazol-3 (2H)-one and 3(2H)-Isothiazolone, 2-Methyl- or 1,2-benzisothiazol-3(2H)-one, and 2-methylisothiazol-3(2H)-one or 1,3-Bis (hydroxymethyl)-5,5-dimethylimidazolidine-2,4-dione and Formaldehyde or 1,2-benzisothiazol-3(2H)-one and 2-Methyl-2H-isothiazol-3-one and N-(3-Aminopropyl)-N-dodecylpropane-1,3 diamine.

In certain embodiments, this freshener for spaces and objects comprises capsules of such characteristics that after the application of the force onto the capsule, the membrane of the fragrance capsule breaks and the fragrance is released at once. In another embodiment, this freshener comprises capsules of such characteristics that after the application of the force onto the capsule, the membrane of the fragrance capsule cracks and, after releasing the initial noticeable amount of the fragrance, it continues to release the fragrance over a certain period of time up to several months. In further embodiment, this freshener for spaces and objects comprises capsules of such characteristics that after the activation of the capsule by touch, the capsule has a better and stronger instantaneous release of the fragrance, but also the effect of prolonged action/fragrance release.

It is possible that in one embodiment all three types of capsules are used, so that this freshener for spaces and objects comprises capsules of such characteristics that after the application of force on the capsule, the membrane of the fragrance capsule cracks and, after releasing the initial noticeable amount of the fragrance, it continues to release fragrance over a certain period of time up to several months and/or after the application of the force onto the capsule, the membrane of the fragrance capsule breaks and the fragrance is released at once and/or after the activation of the capsule by touch, the capsule has a better and stronger instantaneous release of the fragrance, but also the effect of prolonged action/fragrance release.

In one embodiment, instead of the fragrance, the capsule may comprise odour neutralizer, i.e. antiodour, whose function would be, instead of scenting the objects, to neutralize the smell, i.e. the bad odours.

1. Freshener for spaces and objects, comprising a plurality of capsules which contain a fragrant substance, wherein, when a single capsule breaks, the fragrance of the fragrant substance is released into the space and wherein the subject freshener for spaces and objects provides a longer lasting freshness, as the fragrance is protected in each capsule as long as the said capsule remains intact.

2. Freshener for spaces and objects, as claimed in claim 1, characterized in that the fragrant substance comprises demineralised water, a thickener or viscosity modifier, sodium chloride and preservative.

3. Freshener for spaces and objects, as claimed in claim 2, characterized in that the viscosity modifier is a cellulose gum or an acrylic copolymer.

4. Freshener for spaces and objects, as claimed in claim 1, characterized in that the capsule is a nano-capsule.

5. Freshener for spaces and objects, as claimed in claim 1, characterized in that the capsule is produced by the method of nanoencapsulation.

6. Freshener for spaces and objects, as claimed in claim 1 characterized in that the capsule is produced by the method of microencapsulation.

7. Freshener for spaces and objects, as claimed in claim 1, characterized in that the preservative is 5-chloro-2-methyl-4-isothiazolin-3-one and 2-methyl-4-isothiazolin-3-one and benzyl alcohol.

8. Freshener for spaces and objects, as claimed in claim 1, characterized in that the preservative is 1,2-benzisothiazol-3 (2H)-one and 3(2H)-Isothiazolone, 2-Methyl.

9. Freshener for spaces and objects, as claimed in claim 1, characterized in that the preservative is 1,2-benzisothiazol-3(2H)-one and 2-methyl-(2H)-isothiazol-3-one

10. Freshener for spaces and objects, as claimed in claim 1, characterized in that the preservative is 1,3-Bis (hydroxymethyl)-5,5-dimethylimidazolidine-2,4-dione and Formaldehyde.

11. Freshener for spaces and objects, as claimed in claim 1, characterized in that the preservative is 1,2-benzisothiazol-3(2H)-one and 2-Methyl-2H-isothiazol-3-one and N-(3-aminopropyl)-N-dodecylpropane-1,3-di amine.

12. Freshener for spaces and objects, as claimed in claim 1, characterized in that the capsules have such characteristics that after the application of the force onto the capsule, the membrane of the fragrance capsule breaks and the fragrance is released at once.

13. Freshener for spaces and objects, as claimed in claim 1, characterized in that the capsules have such characteristics that after the application of the force onto the capsule, the membrane of the fragrance capsule cracks and, after releasing the initial noticeable amount of the fragrance, it continues to release the fragrance over a certain period of time up to several months.

14. Freshener for spaces and objects, as claimed in claim 1, characterized in that the capsules have such characteristics that after the capsule is activated by touch, the capsule has a better/stronger instantaneous release of the fragrance, but also the effect of prolonged action/fragrance release.

15. Freshener for spaces and objects, as claimed in claim 1, characterized in that the capsules have such characteristics that after the application of the force onto the capsule, the membrane of the fragrance capsule cracks and, after releasing the initial noticeable amount of the fragrance, it continues to release the fragrance over a certain period of time up to several months and/or that after the application of the force onto the capsule, the membrane of the fragrance capsule breaks and the fragrance is released at once and/or that after the capsule is activated by touch, the capsule has a better/stronger instantaneous release of the fragrance, but also the effect of prolonged action/fragrance release.

16. Freshener for spaces and objects, as claimed in claim 1, characterized in that it is used for freshening of the fabrics and textiles.

17. A method of using the freshener for spaces and objects, of claim 1, characterized in that it is applied by spraying directly onto the surface or the fabric.

18. A method of using the freshener for spaces and objects, of claim 1, characterized in that it is applied by sprinkling onto the surface.
